(19) United States
(12) Patent Application Publication
(10) Pub. No.: US 2026/0201406 A1
(43) Pub. Date: Jul. 16, 2026

(54) TOMATO PLANT HAVING IMPROVED WHITEFLY RESISTANCE

(71) Applicant: Enza Zaden Beheer B.V, Enkhuizen (NL)

(72) Inventors: Teresa Montoro Ponsoda, Roquetas de Mar (ES); Jan-Willem De Kraker, Enkhuizen (NL); Juan David Cano Martinez, Roquetas de Mar (ES); Marieke Ykema, Harlingen (NL)

(21) Appl. No.: 19/129,917

(22) PCT Filed: Nov. 15, 2022
(86) PCT No.: PCT/EP2022/082012

§ 371 Date: May 14, 2025

(51) Int. Cl. C12N 15/82; C07K 14/415; C12N 9/10

(52) U.S. Cl. CPC C12N 15/8286; C07K 14/415; C12N 9/1029; C12N 15/8245; C12Y 203/01

(57) ABSTRACT

The present invention relates to a tomato plant having improved insect resistance, more specifically whitefly or mite resistance, wherein the plant comprises a ASAT3 gene encoding for an acetyl-CoA-dependent acyltransferase enzyme and an AP2e gene encoding for a APETALA2 ethylene-responsive transcription factor. The present invention further relates to methods for providing a tomato plant having improved insect resistance and the use of a ASAT3 gene in combination with an AP2e gene for providing insect resistant tomato plants.

The present invention relates to a tomato plant having improved insect resistance, more specifically whitefly resistance, wherein the plant comprises a ASAT3 gene encoding for an acetyl-CoA-dependent acyltransferase enzyme and an AP2e gene encoding for a APETALA2 ethylene-responsive transcription factor. The present invention further relates to methods for providing a tomato plant having improved insect resistance and the use of a ASAT3 gene in combination with an AP2e gene for providing insect resistant tomato plants.

Economically, whiteflies is one of the most important pest of tomato crops. It causes direct damage through feeding on plant phloem sap and indirect damage through virus transmission and sooty mold growth. Whitefly related damage reduces crop quality and quantity, and it can cause a reduction of plant vigour and yield, early wilting, leaf chlorosis and defoliation.

Whiteflies are of the family Aleyrodidae that typically feed on the abaxial side of plant leaves. There are more than 1500 species described and especially in warm or tropical climates and in greenhouses, whiteflies present a major problem in crop protection in warm climates and crops grown under greenhouse conditions, resulting in huge economic losses annually worldwide. Many whitefly species are very small in size which complicates their control in greenhouses, and when leaving unchecked whitefly populations in greenhouses rapidly become overwhelming. Whitefly related damage reduces crop quality and quantity, such as a reduction of plant vigour and yield, early wilting, leaf chlorosis and defoliation. The silverleaf whitefly (Bemisia tabaci) is one species of whitefly that are currently one of the most important agricultural pests.

Although several species of whitefly may cause some crop losses simply by sucking sap when they are very numerous, the major harm they do is indirect. The major importance as crop pests is their role as vectors and in the transmission of diseases of plants. Virus transmission is the main problem caused by whiteflies. Whiteflies are vectors of more than 200 plant viruses among them the most relevant ones belong to the Begomovirus genus (TYLCV and others) and also various viruses in the Crinivirus (e.g. ToCV) and Torrado virus (ToANV, ToTV). Furthermore, whiteflies feed by tapping into the phloem of plants, introducing toxic saliva and decreasing the plants' overall turgor pressure. The whitefly secretes large amounts of honeydew that support harmful infestations of fungi like sooty mold. Since whiteflies congregate in large numbers, susceptible plants can be quickly overwhelmed.

Insecticides such as neonicotinoid, organochlorines, and organophosphate compounds are widely used and are an effective way to control whiteflies. However, continuous application of insecticides results in the development of whiteflies becoming resistant. In addition, more environmentally friendly, biological methods have also been proposed to control whitefly infestation, such as using natural predators and parasitoids (e.g. using green lacewing larvae) to control whitefly infestations, or washing of the plants to reduce the number of the pests on the plants. However, also these methods do not provide an optimal solution to the pests, and whitefly remains difficult to control.

Especially in tomato (Solanum lycopersicum) and pepper (Capsicum spp.) whitefly infestations prove to be a problem. Tomato is classified as Solanum sect. Lycopersicon comprising 13 species, of which Solanum lycopersicum is the cultivated tomato, whereas the other 12 species are wild relatives. The genus Capsicum has 25 species of which five are cultivated, including C. annuum, C. chinense, C. baccatum, C. pubescens and C. frutescens. The domestication of tomato and pepper resulted in loss of genetic diversity which makes them prone to abiotic and biotic stresses such as pest attacks. To date, no cultivated tomato and sweet peppers are resistant to whiteflies. Previously several studies have been performed to find whitefly resistance in wild relatives of tomato and sweet pepper. Several wild relatives of tomato (S. pennellii, S. habrochaites, S. peruvianum and S. pimpinellifolium) are known to be more resistant than the cultivated material. Resistance to whitefly could contribute to a higher level of virus resistance and a higher durability of the viruses' resistance genes in use due to the lower selection pressure on the virus resistance genes.

Antibiosis is one of the resistance mechanisms in which a plant exerts an adverse influence on the growth and survival of the insect. One of the most prominent tomato characters that contribute to whitefly antibiosis are trichomes, which are fine outgrowths or appendages on plants, such as glandular hairs. Their function is to secrete metabolites for the plant, including terpenoids, phenylpropanoids, flavonoids, methyl ketones and acyl sugars having diverse functions in the plant related to growth and development, and stress response. For example, mono- and sesquiterpenes, methyl ketones and acyl sugars are secondary metabolites that are known to be associated with whitefly resistance in tomato. Although glandular trichomes seem to play an important role in whitefly resistance, it is the compounds within the trichomes that are decisive. A high correlation was found between presence of specific trichomes (type IV trichomes) and whitefly resistance. Previous studies showed that whitefly resistance is based on several mechanisms involving many genes which makes it a complex trait. Efforts of introducing whitefly resistance in the cultivated tomato were not successful and new approaches and resistant sources should be considered.

Considering the above, there is a need in the art for tomato plants having an improved insect resistance. In addition, there is a need in the art for a method for providing plants having improved insect resistance, more specifically tomato plants having improved resistance against whiteflies.

It is an object of the present invention, amongst other objects, to address the above need in the art. The object of present invention, amongst other objects, is met by the present invention as outlined in the appended claims.

Specifically, the above object, amongst other objects, is met, according to a first aspect, by the present invention by a tomato plant having improved whitefly resistance, wherein said plant comprises a combination of an acetyl-CoA-dependent acyltransferase gene (ASAT3) encoding a cDNA sequence having at least 95%, preferably at least 98%, more preferably at least 99% sequence identity with SEQ ID No. 1, and an APETALA2e ethylene-responsive transcription factor gene (AP2e) encoding a cDNA sequence having at least 95%, preferably at least 98%, more preferably at least 99% sequence identity with SEQ ID No. 3, wherein said combination of ASAT3 and AP2e genes result in an increased acyl sugar content of one or more sugars selected from the group consisting of C₃₉H₆₆O₁₅, C₃₉H₆₈O₁₅, C₃₈H₆₆O₁₅, C₄₀H₇₀O₁₅, preferably all of said sugars, as compared to a tomato plant not comprising said combination of genes. Even more preferably the ASAT3 gene encodes a cDNA of SEQ ID No.2 and the AP2e gene encodes a cDNA of SEQ ID. No.4. Experiments show that the introgression and combination of the ASAT3 and AP2e genes into commercial tomato (S. lycopersicum) contributes to the production of specific acylsucroses that have a clear impact on the whitefly toxicity resulting in an increased whitefly resistance in the tomato plant. Correlation analysis between individuals a-cylsucroses and whitefly mortality revealed that different individual acylsucrose molecules play a different role in the resistance of the tomato plants to the whiteflies. When comparing whitefly mortality versus individual acylsucroses, specific acylsucrose compounds showed a coefficient correlation higher than 0.5 and therefore having a higher impact on resistance; C₃₉H₆₆O₁₅, C₃₉H₆₈O₁₅, C₃₈H₆₆O₁₅, and C₄₀H₇₀O₁₅. Experiments of the bioassay in tomato plant according to the present invention show that at the identified acyl sugar concentrations of the specifically indicated acyl sugars, the level of resistance, i.e. increased WF resistance is observed of a whitefly mortality of at least 50%, preferably at least 55%, more preferably at least 60%, even more preferably at least 65%. Plants that do not comprise the ASAT3 and/or AP2e gene in its genome did not show the identified acyl sugar concentrations of the specifically indicated acyl sugars, and this level of resistance.

The tomato plant of present invention, preferably a S. lycopersicum plant has improved insect resistance, wherein the plant comprises an ASAT3 gene in combination with and an AP2e gene. The APETALA2 (AP2) gene family (sometimes also called the AP2/ethylene-responsive element-binding factor (ERF) gene family or ERF/AP2 gene family) defines a large gene family (>100+ genes) of DNA-binding proteins called AP2/ERF in tomato plants. The AP2 genes perform an array of functions including hormone regulation, the establishment of the floral meristem organ identity, and regulation and growth of floral organs and development, as well as various responses to environmental stimuli and stress responses. Furthermore, it is known that various different AP2 genes provide for changes in the ratio of hexose to sucrose during seed development of a plant, and that the AP2 protein regulates the quantity of sugars in the system and is involved in transportation, shaping, and signalling in said plant using these various sugars. Surprisingly it was found that the ASAT3 gene in combination with the AP2e gene promotes and regulates the production of the type of specific S4 type acyl sugars C₃₉H₆₆O₁₅ (S4:dC27 acyl sucrose), C₃₉H₆₈O₁₅ (S4:C27 acyl sucrose), C₃₈H₆₆O₁₅ (S4:C26 acyl sucrose), and C₄₀H₇₀O₁₅ (S4:C28 acyl sucrose), and that these specific increased production of acyl sugars are linked with high levels of insect resistance in the plant. The ASAT3 gene encodes for an acyl-CoA-dependent acyltransferase (type III) enzyme capable of acylating the furanose ring of diacil-sucrose. ASAT3 seems to be involved in the last step of acylsugar pathway catalysing the acylation of tri-acyl sucroses to produce a particular set of tetra-acyl sucroses.

The AP2e gene encode for APETALA2e ethylene-responsive transcription factor that is involved in the regulation of acyl sugars being produced. It seems that AP2e is related to the capacity of producing general amounts of different types of acyl sugars, while the ASAT3 promotes the production of the specific S4 type acyl sugars that impact the insect resistance in the plant. The AP2e affects the total amount of acyl sucrose produced by switching on genes involved in the biosynthetic pathway and formation of trichomes, whereas ASAT3 seems to catalyse or exert the final step of the acylsugar pathway, more specifically converting S3 into the S4 type. An active ASAT3 enzyme seems to be responsible for adding an additional acyl-group to acyl sucrose that has already three acyl-groups. This results in an increase of the amount of specific tetra-acyl sucroses, which results in an improved insect resistance as observed in tomato plants of present invention. When the ASAT3 gene is absent in the tomato plant, plants showed a reduced resistance to whitefly in comparison to tomato plant that comprised the ASAT3 gene.

Experiments show that the type of acyl sugars is crucial for providing the whitefly resistance in tomato plants. It was observed that plants comprising the AP2e gene and producing acyl sugars are not always resistant to whitefly. However, in case the plant also comprises the ASAT3 gene, the plant shows improved insect resistance which was linked to increased levels of specific tetra-acyl (S4) sucroses, more specifically C₃₉H₆₆O₁₅, C₃₉H₆₈O₁₅, C₃₈H₆₆O₁₅, and C₄₀H₇₀O₁₅, in comparison to susceptible plants.

The tomato plant of present invention has increased S4 acyl sugar content in comparison to a plant that does not comprise the ASAT3 gene in combination with the AP2e gene. The acyl sugars are trichome exudates, more specifically the C₃₉H₁₆O₁₅ (S4:dC27 acyl sucrose), C₃₉H₆₈O₁₅ (S4:C27 acyl sucrose), C₃₈H₆₆O₁₅ (S4:C26 acyl sucrose), and C₄₀H₇₀O₁₅ (S4:C28 acyl sucrose) acyl sugars are type IV trichrome exudates, that cause resistance to insects in tomato plants. AP2e is involved in both trichome development and acyl sugar production. Tomato plants comprising the AP2e gene have an increase of type IV trichomes in the leaf surface and stem. Many trichome exudates of tomato (approx. 90%) are comprised of acyl sugars, of which more than 70 compounds are known. The acyl sugars produced in tomato consist of different combinations of acyl groups, originating from different aliphatic acids with varying chain length and esterified to the hydroxyl groups of glucose or sucrose. The acyl chains are primarily of short to medium chain length aliphatic acids with either a branched or strait chain. In tomato it has been shown that the primary short acyl chains of acyl sugar are either acetate (C2) or branched chain amino acid derived, i.e. 2-methyl-propanoic acid (C4) and 3-methyl-butanoic acid (C5). Longer acyl groups are probably derived from beta-oxidation products of fatty acids. However, the presence and abundance of specific acyl sugars differ significantly between resistant and susceptible plants, wherein specifically C₃₉H₆₆O₁₅ (S4:dC27 acyl sucrose), C₃₉H₆₈O₁₅ (S4:C27 acyl sucrose), C₃₈H₆₆O₁₅ (S4:C26 acyl sucrose), and C₄₀H₇₀O₁₅ (S4:C28 acyl sucrose) acyl sugar content is high in insect resistant plants. These acyl sugars are sticky substances that act as a glue trap and are also toxic for insects, more specifically whiteflies, providing improved insect resistance for the plant. Furthermore, not only whiteflies but other pierce-sucking insects avoid settling on leaves in the presence of these specific acyl sugars.

According to a preferred embodiment, the present invention relates to the tomato plant, wherein the whitefly is one or more selected from the group consisting of Aleurocanthus woglumi (citrus blackfly), Aleyrodes proletella (cabbage whitefly), Bemisia tabaci (silverleaf whitefly), Trialeurodes vaporariorum (greenhouse whitefly), preferably Trialeurodes vaporariorum and/or Bemisia tabaci.

According to a preferred embodiment of the present invention the present plants detailed above are not plants exclusively obtained by means of an essentially biological process.

Although the present genomic regions or fragments can be introduced into tomato plants by introgression, because the nucleotide sequences of the present genomic fragments are known, these genomic fragments, for example, can be artificially constructed in yeast and subsequently allowed to recombine with susceptible tomato genomes. Alternatively, these genomic regions or fragments can be amplified by long-range PCR amplifications and the resulting amplification fragments can be transformed into plant cells in a single step or in a series of transformations ultimately resulting in the present tomato plants. The present genomic fragments, completely or in parts later to be reassembled, can also be isolated from gels or columns for example after restriction digestion, and subsequently transformed into tomato cells. Furthermore, mutations, deletions or insertions in the genome can be obtained via EMS mutagenesis, and/or CRISPR technology. Yet alternatively, the genomic fragments of interest can be introduced into a vector under a (strong) promotor. Subsequently, susceptible plants can be transformed with the vector and the sequence of interest would be expressed resulting in resistance. These techniques are readily available for the skilled person. Construction of artificial chromosomes comprising the present genomic fragments is also contemplated within the context of the present invention.

According to yet another preferred embodiment, the present invention relates to the tomato plant, wherein the ASAT3 gene encodes for the protein sequence represented by SEQ ID No. 2, and wherein the AP2e gene encodes for the protein sequence represented by SEQ ID No. 4.

According to yet another preferred embodiment, the present invention relates to the tomato plant, wherein the acyl sugar content of C₃₉H₆₆O₁₅, C₃₉H₆₈O₁₅, C₃₈H₆₆O₁₅, and C₄₀H₇₀O₁₅ together is at least 200 μg/g of fresh weight (FW) of plant leaves, preferably at least 250 μg/g of fresh weight (FW) of plant leaves, more preferably at least 300 μg/g of fresh weight (FW) of plant leaves. The fresh weight (FW) is the weight of a plant or plant part, in this case plant leaves when harvested. Experiments of the bioassay in tomato plant according to the present invention show that at the identified acyl sugar concentrations of the specifically indicated acyl sugars, the level of resistance is observed of a whitefly mortality of at least 50%, preferably at least 55%, more preferably at least 60%, even more preferably at least 65%.

According to another preferred embodiment, the present invention relates to the tomato plant, wherein the acyl sugar content of C₃₉H₆₆O₁₅ is at least 1 μg/g of FW of plant leaves, preferably at least 1.5 μg/g of FW of plant leaves, more preferably at least 2 μg/g of FW of plant leaves. Although C₃₉H₆₆O₁₅ is present in small amounts in view of the other identified acyl sugars in the tomato plant showing increased WF resistance, this specific acyl sugar seems to provide or contribute the most on the toxicity effect on WF and contributing to the resistance in the plants.

According to a preferred embodiment, the present invention relates to the tomato plant, wherein the acyl sugar content of C₃₉H₆₈O₁₅ is at least 200 μg/g of FW of plant leaves, preferably at least 250 μg/g of FW of plant leaves, more preferably at least 300 μg/g of FW of plant leaves.

According to another preferred embodiment, the present invention relates to the tomato plant, wherein the acyl sugar content of C₃₈H₆₆O₁₅ is at least 5 μg/g of FW of plant leaves, preferably at least 10 μg/g of FW of plant leaves, more preferably at least 15 μg/g of FW of plant leaves.

According to yet another preferred embodiment, the present invention relates to the tomato plant, wherein the acyl sugar content of C₄₀H₇₀O₁₅ is at least 5 μg/g of FW of plant leaves, preferably at least 10 μg/g of FW of plant leaves, more preferably at least 15 μg/g of FW of plant leaves.

According to yet another preferred embodiment, the present invention relates to the tomato plant, wherein the plant is obtainable from deposit NCIMB 44054. Most preferably the present invention relates to the tomato plant, wherein the AP2e gene and ASAT3 gene is obtainable, derived, or originates from a tomato plant deposited at NCIMB Ltd, Aberdeen, Scotland on 14 Oct. 2022, under number NCIMB 44054.

According to another preferred embodiment, the present invention relates to the tomato plant, wherein the plant is furthermore resistant to mite, preferably spider mite (Tetranychus urticae).

According to another preferred embodiment, the present invention relates to the tomato plant, wherein the acetyl-CoA-dependent acyltransferase gene (ASAT3) is at least heterozygous present in the genome of the plant, preferably homozygous.

According to another preferred embodiment, the present invention relates to the tomato plant, wherein the APETALA2e ethylene-responsive transcription factor gene (AP2e) is at least heterozygous present in the genome of the plant, preferably homozygous. Experiments show that when the plant comprises both AP2e and ASAT3 homozygous in the genome, the whitefly resistance seems to be most optimal. However, plant comprising a ASAT3 gene heterozygous also show to be resistant to whitefly.

According to a preferred embodiment, the present invention relates to the tomato plant, wherein said plant is a Solanum lycopersicum var. cerasiforme.

According to another preferred embodiment, the present invention relates to the tomato plant, wherein said plant does not comprise a SlAT2 gene in its genome encoding a cDNA sequence with SEQ ID No. 5.

According to yet another preferred embodiment, the present invention relates to the tomato plant, wherein the combination of ASAT3 and AP2e genes furthermore result in an increased acyl sugar content of C₃₂H₅₄O₁₅ as compared to a tomato plant not comprising said combination of genes. In case the plant comprises the AP2e and ASAT3 gene, but does not comprise the SlAT2 gene, apart from the previous four identified sugars, also C₃₂H₅₄O₁₅ seems to contribute to the increased WF resistance phenotype of the tomato plant of present invention.

The present invention, according to a second aspect, relates to seeds, fruits or plant part of a tomato plant of present invention.

The present invention, according to a further aspect, relates to a method for providing a tomato plant having improved whitefly resistance, the method comprises the steps of providing a whitefly susceptible tomato plant and mutating its genome comprising;
providing a combination of an acetyl-CoA-dependent acyltransferase gene (ASAT3) encoding a cDNA sequence having at least 95% sequence identity with SEQ ID No. 1, and an APETALA2e ethylene-responsive transcription factor gene (AP2e) encoding a cDNA sequence having at least 95% sequence identity with SEQ ID No. 3, wherein said combination of ASAT3 and AP2e genes result in an increased C₃₉H₆₆O₁₅ (S4:dC27 acyl sucrose), C₃₉H₆₈O₁₅ (S4:C27 acyl sucrose), C₃₉H₆₆O₁₅ (S4:C26 acyl sucrose), C₄₀H₇₀O₁₅ (S4:C28 acyl sucrose) and C₃₂H₅₄O₁₅ (S4:C20 acyl sucrose) acyl sugar content as compared to a tomato plant not comprising said combination of genes.

The present invention, according to a further aspect, relates to a method for providing a tomato plant having improved whitefly resistance, wherein the method comprises the steps of;
a) crossing of a tomato plant that is susceptible to whitefly with a tomato plant as disclosed above,
b) selecting S. lycopersicum plants having improved insect resistance that comprise the AP2e gene and ASAT3 gene.

According to a preferred embodiment, the present invention relates to the method for providing a tomato plant having improved whitefly resistance, wherein presence of the AP2e gene in said S. lycopersicum plants having improved insect resistance is determined by using markers SEQ ID No. 7 and SEQ ID No. 8, and wherein presence of the ASAT3 gene is determined by using markers SEQ ID No. 9 and SEQ ID No. 10. Furthermore, selection of whitefly resistant tomato plants can also be done by determination or identification of the specific sequences (cDNA, or protein sequences) of ASAT3 and AP2e, as identified herein as SEQ ID NO. 1, 2, 3 and 4, respectively.

According to another preferred embodiment, the present invention relates to the method for providing a tomato plant having improved whitefly resistance, wherein the selection of S. lycopersicum plants having improved insect resistance is by determination of C₃₉H₆₈O₁₅, C₃₉H₆₈O₁₅, C₃₈H₆₆O₁₅, and/or C₄₀H₇₀O₁₅ acyl sugar content, wherein the acyl sugar content of C₃₉H₆₆O₁₅, C₃₉H₆₈O₁₅, C₃₈H₆₆O₁₅, and C₄₀H₇₀O₁₅ together is at least 200 μg/g of fresh weight (FW) of plant leaves, and/or of C₃₉H₆₆O₁₅ is at least 1 μg/g of FW of plant leaves, and/or of C₃₉H₆₈O₁₅ is at least 200 μg/g of FW of plant leaves, and/or of C₃₈H₆₆O₁₅ is at least 5 μg/g of FW of plant leaves. Apart from determining the presence of the AP2e and ASAT3 genes in the tomato plants, selection of tomato plants having improved insect resistance can be based on the determination of C₃₉H₆₆O₁₅, C₃₉H₆₈O₁₅, C₃₈H₆₆O₁₅, and C₄₀H₇₀O₁₅ acyl sugar content.

The present invention, according to a further aspect, relates to a method for providing a tomato plant having improved whitefly resistance, wherein the method comprises the steps of
a) providing a tomato plant as described herein comprising the AP2e and ASAT3 genes,
b) crossing the tomato plant of step a) with a tomato plant that is more susceptible to whitefly not comprising the AP2e and ASAT3 genes,
c) optionally, selfing the plant obtained in step b) for at least one time,
d) selecting the plants having improved whitefly resistance.

The present invention, according to a further aspect, relates to a combination of an acetyl-CoA-dependent acyltransferase gene (ASAT3) encoding a cDNA sequence having at least 95% sequence identity with SEQ ID No. 1, and an APETALA2e ethylene-responsive transcription factor gene (AP2e) encoding a cDNA sequence having at least 95% sequence identity with SEQ ID No. 3 for providing insect resistance in tomato plants.

The present invention, according to a further aspect, relates to the use of a combination of the two genes AP2e and ASAT3 as defined above in tomato plants for providing whitefly resistant tomato plants.

The present invention will be further detailed in the following examples and figures wherein:

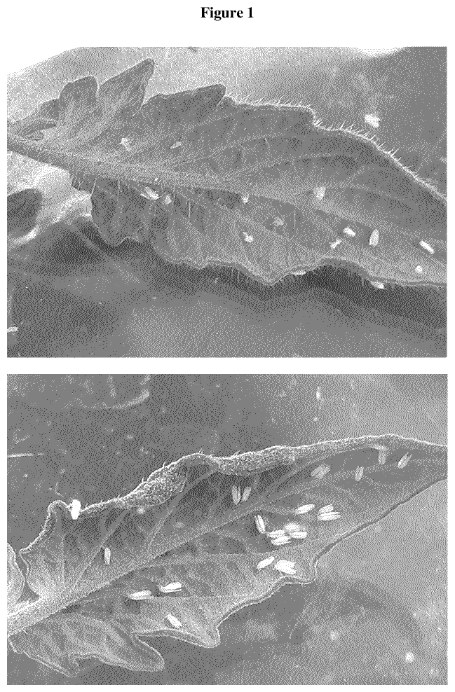

FIG. 1: Shows the leave of a tomato plant (S. lycopersicum) according to present invention (upper panel) and the leave of an insect susceptible tomato plant (S. lycopersicum) (lower panel). Both tomato plants have been exposed to in vitro whitefly infestation in a glass petri dish. The leaves of the plants of present invention are free from alive whitefly infestation; most whiteflies are deformed and dead. In contrast to the leaves of the insect susceptible tomato plant, which are clearly infected by whitefly that are alive and having a healthy shape.

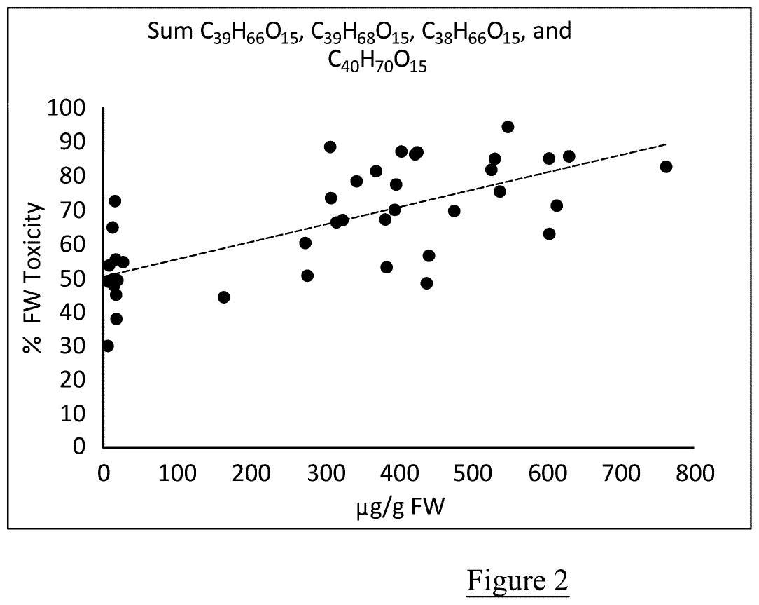

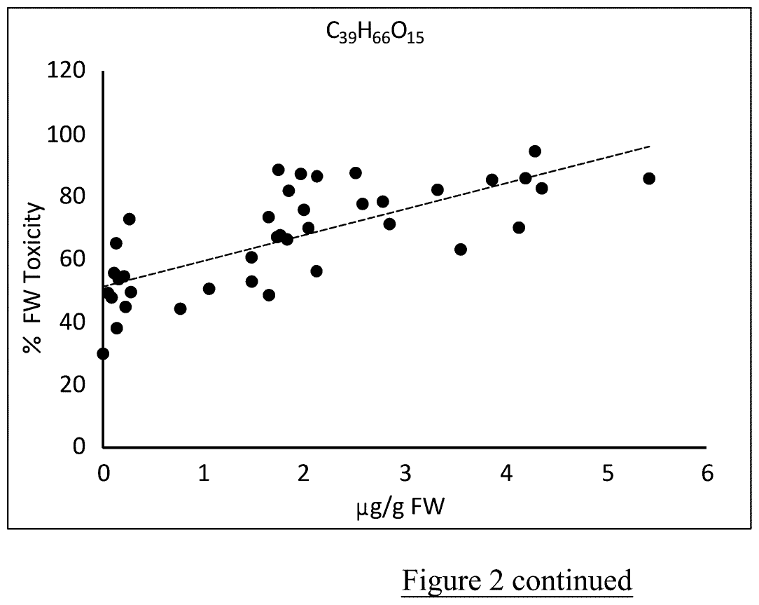

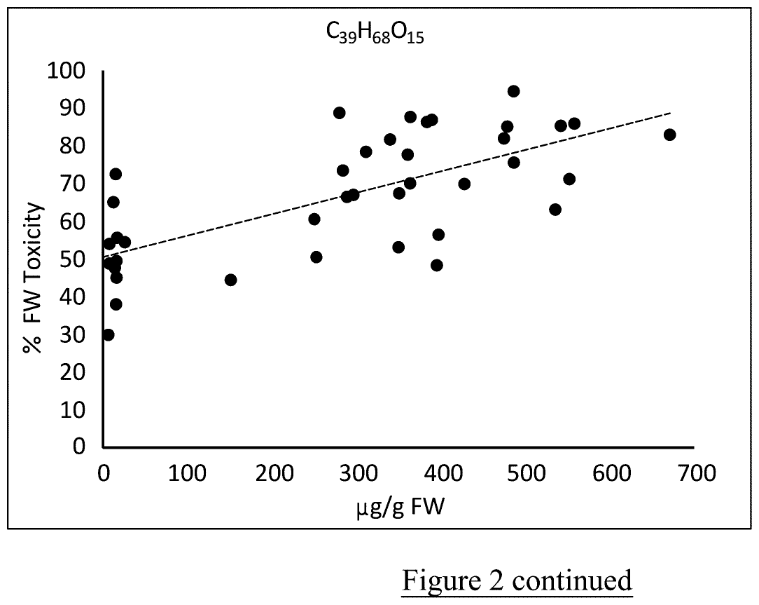

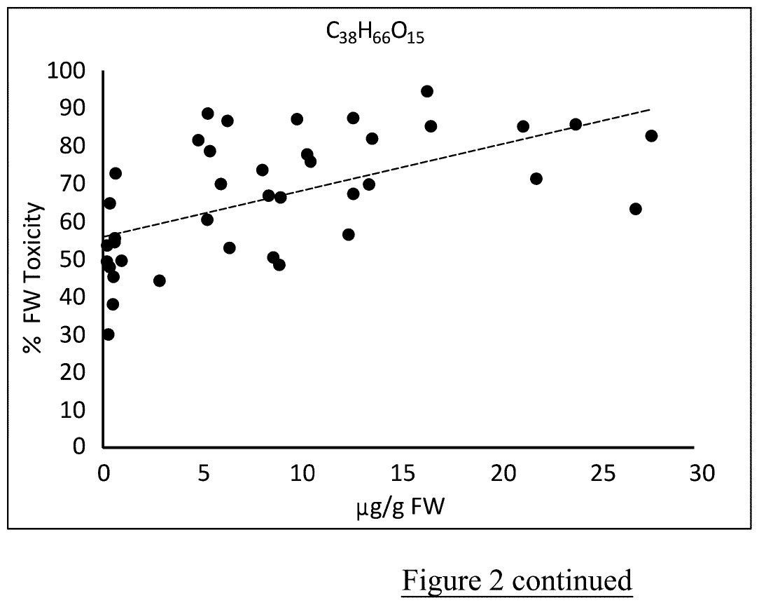

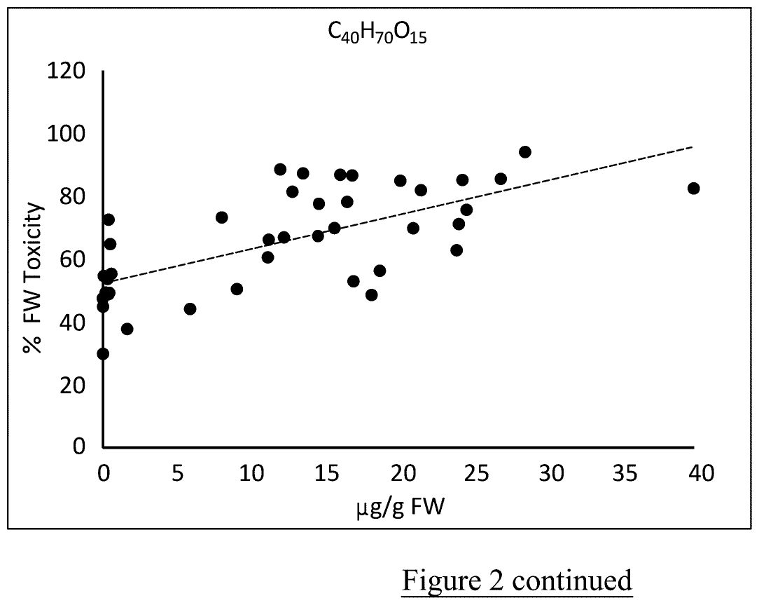

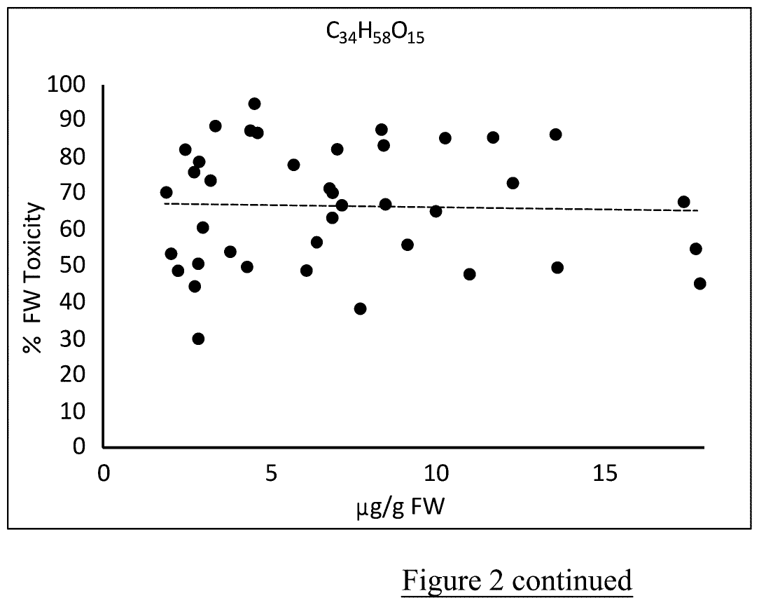

FIG. 2: Shows the percentage of dead whiteflies (WF) against increasing concentrations of acyl sugar in a tomato plant. The graphs shows that there is a correlation between the number of dead whiteflies and the concentration of acyl sugars C₃₉H₆₆O₁₅, C₃₉H₆₈O₁₅, C₃₈H₆₆O₁₅, and C₄₀H₇₀O₁₅. These specific acyl sugars show to negatively affect the survival of the whiteflies. On the other hand, other acyl sugars present in the plant, such as C₃₄H₅₈O₁₅ do not affect the whiteflies. The fresh weight (FW) is the weight of a plant, in this case FW of plant leaves when harvested. The bioassay shows that the level of acyl sugar content in the leaves of the plant directly affects the level of resistance that is observed as whitefly mortality.

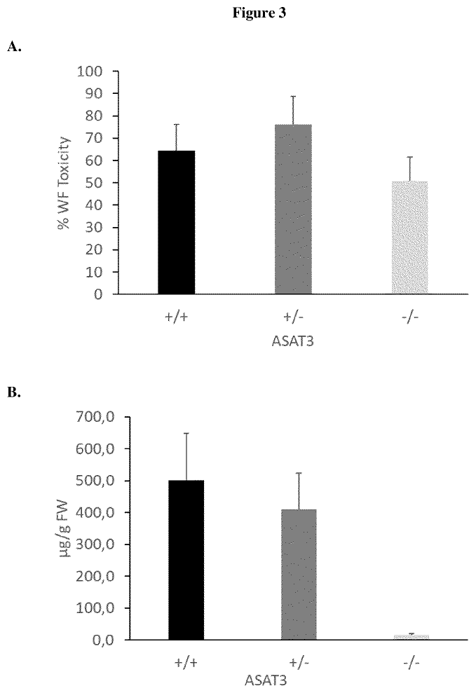

FIG. 3: Shows the average whitefly (WF) toxicity level (FIG. 3A) and average amount of C₃₉H₆₆O₁₅, C₃₉H₆₈O₁₅, C₃₈H₆₆O₁₅, and C₄₀H₇₀O₁₅ acyl sugar (FIG. 3B, and indicated as S4 type acyl sugar in μg/gFw of plant) determined in tomato plants that comprise the ASAT3 gene homozygous (+/+) or heterozygous (+/−), or in case the ASAT3 gene is absent (−/−). The absence/presence of ASAT3 gene is directly linked to the WF toxicity levels and the amount of said specific sugars in the tomato plant. All plants comprised the AP2e gene homozygous. Plants are considered WF resistant when the observed WF toxicity is above 50%, more preferably above 60% WF toxicity levels.

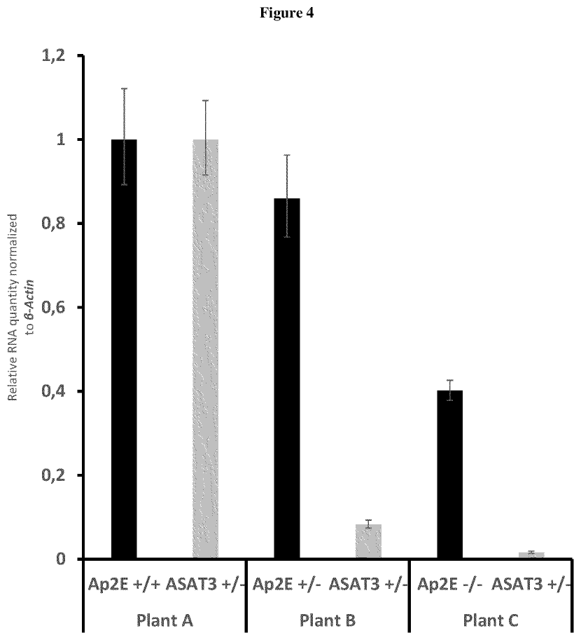

FIG. 4: Show the AP2e and ASAT3 gene expression as determined by RT PCT using β-Actin as endogenous control. Expression values of each gene were normalized to the endogenous β-Actin gene AP2e and ASAT3 gene expression levels were determined in whitefly resistant and susceptible tomato plants. Three types of plants were included; Plant A is homozygous (+/+) for the AP2e gene and heterozygous (+/−) for the ASAT3 gene, Plant B is heterozygous for both AP2e and ASAT3, Plant C is absent (−/−) of AP2e and heterozygous for ASAT3. The presence/absence of the AP2e gene correlates with the expression of ASAT3 gene and the WF toxicity levels and the amount of specific sugars in the tomato plant.

### EXAMPLES

### Detached Leaves Bioassay

A young leaflet from the third leaf were detached from at least 12-week-old tomato plants. Next, the petiole of the leaflet was placed into a tube filled with nutritive agar to avoid dehydration. Later, the tube is placed (using blue-tack) in the middle of the lid of a glass petri dish such that the adaxial side faces up and the abaxial side faces down leaving enough space between the leaf and the petri dish. Each petri dish was then inoculated with 25 whiteflies, which were anaesthetized with CO₂ for 3 seconds.

After 24-48 hours, the number of whiteflies in the adaxial or abaxial surface was counted together with the number of dead whiteflies. Each plant was tested twice and the number of whiteflies that were alive (feeding from the adaxial and the abaxial part of the leaf plus the whiteflies still flying around in the petri dish) versus the dead whiteflies were used to calculate the percentage of dead whiteflies. Correlation analysis between specific acyl sucroses and the whitefly mortality revealed that different specific acyl sucrose molecules play a different role in the resistance/susceptibility to whiteflies.

As showed in FIG. 2 representing whitefly (WF) mortality versus the specific acyl sucrose content present in the plant, mainly C₃₉H₆₆O₁₅ (S4:dC27 acyl sucrose), C₃₉H₈₀₁₅ (S4:C27 acyl sucrose), C₃₈H₆₆O₁₅ (S4:C26 acyl sucrose), and C₄₀H₇₀O₁₅ (S4:C28 acyl sucrose) acyl sugars showed have a high impact on resistance. No such significant relation was for example observed in the acyl sugar content of C₃₇H₆₄O₁₅ (S4:C25), C₃₂H₅₆O₁₄ (S3:C20), C₃₄H₅₈O₁₅ (S4:C22) in insect susceptible and resistant plants.

### Liquid Chromatography Mass Spectrometry (LC-MS) Analysis Acyl Sugar Content in Tomato

A chemical analysis by LC-MS of the leaf surface was performed on a set of tomato plants, including insect resistant tomato plants according to present invention, intermediate resistant plants, and susceptible plants (all S. lycopersicum). Plants were grown till 10 side shoots and one punch of a leaflet from the third leaf was taken with a 15-ml tube (1.5 cm of diameter) by opposing the cap and the opening on both sides of the leaflet surface. Then, the punch is submerged in 2 ml of methanol plus sucrose octoacetate (10 mg/l) and shaken for 15 seconds. Next, the leaflet punch is taken out and 600 ul are transferred to 0.5 ml 96-well plate. The methanol is evaporated, the sample redissolve in 300 uL methanol and analyzed using an Agilent 1290 Infinity II UHPLC coupled to an Agilent 6230 TOF mass spectrometer.

One μL of extract was injected and separated on an Agilent ZORBAX RRHD Eclipse Plus C18 column at 50° C. with a mobile-phase flow-rate of 0.3 mL/min. The mobile phase was comprised of water+0.1% formic acid (A) and acetonitrile+0.1% formic acid (B) in the following A:B gradient; from 60:40 to 45:55 in 6 minutes to 10:90 in 8 minutes to 60:40 in 3 minutes. Molecules were ionised at 325 eV (positive mode) and detected in a range of 50-1500 mu at 1 spectrum/second. The extract comprised mainly of acylsugars that were detected as sodium adducts in the mass spectrometer.

Individual acyl sugars were identified using MassHunter Qualitative Analysis software (Agilent) by calculating the molecular formulas on the basis of the sodium and potassium adducts constituting the chromatographic peaks. Here, molecular formulas were constrained by allowing carbon, hydrogen and oxygen atoms to form the molecule that, pairs with H⁺, Na⁺ and K⁺ in the adduct, plus a double bond equivalent (DBE) range of 1-10. The exact mass of the adduct ion in combination with the DBE allows extrapolation of the basic structure of the acyl sugar molecule; the backbone moiety, number of acyl chains and the total number of carbon atoms forming the acyl chains. Amounts of acylsugars were calculated by using MassHunter Quantitative Analysis Software (Agilent) for chromatogram peak integration and comparing the total peak area of the individual acylsugars to that of the internal standard (sucrose octaacetate).

Corresponding results were obtained with the LC-MS, as was also obtained with the detached leaves bioassay as described above, that specific acyl sugars are involved in the insect resistance. The previously indicated S4 type acyl sugar compounds are present in high concentrations in plants showed to be highly resistant to whiteflies. In contrast in plants that showed to be susceptible to whitefly, no or only low concentrations of these specific acyl sugars were detected by LC-MS. From the LCMS results, it is concluded that plants having improved insect resistance is linked to high C₃₉H₆₆O₁₅ (S4:dC27 acyl sucrose), C₃₉H₆₈O₁₅ (S4:C27 acyl sucrose), C₃₈H₆₆O₁₅ (S4:C26 acyl sucrose), and C₄₀H₇₀O₁₅ (S4:C28 acyl sucrose) acyl sugars. No significant changes were observed in the acyl sugar content of C₃₇H₆₄O₁₅ (S4:C25), C₃₂H₅₆O₁₄ (S3:C20), C₃₄H₅₈O₁₅ (S4:C22) in insect susceptible and resistant plants.

### Genotypic Analysis, Mapping of ASAT3 and AP2e.

The production of acyl sugars in tomato plants is linked with high levels of insect resistance in the plant. Important is to know which type of acyl sugars are needed for insect resistance. Looking into genotypic data on the resistant tomato plant population (S. lycopersicum) by marker analysis, marker M1 and M5 (Table 1) were used to identify the QTLs that clearly correlate with the production of C₃₉H₆₆O₁₅ (S4:dC27 acyl sucrose), C₃₉H₆₈O₁₅ (S4:C27 acyl sucrose), C₃₈H₆₆O₁₅ (S4:C26 acyl sucrose), and C₄₀H₇₀O₁₅ (S4:C28 acyl sucrose) acyl sucrose.

Briefly, genomic regions that are linked to the amount of acyl sugars which are produced by type IV trichomes have been mapped on chromosome 6, based on the reference genome SL2.40. It was determined that the region involved in acyl sugar production linked to insect resistance is located between positions 43250794 bp and 43259933 bp. Marker M5 is 100% linked with the amount of acyl sugars being produced. Based on the reference genome SL2.40 and in silico prediction analysis (ITAG 2.3), one gene Solyc06g075510.2 is located in the fine mapped region that encodes for an APETALA2 ethylene-responsive transcription factor (AP2e).

Furthermore, the type of acyl sugars is crucial for providing the whitefly resistance in tomato plants. It was observed that plants comprising the AP2e gene and producing acyl sugars are not always resistant to whitefly. Comparing the acyl sugar profiles of the susceptible and resistant plants, it was concluded that plants having improved insect resistance are linked to high levels of tetra-acyl (S4) sucroses C₃₉H₆₆O₁₅ (S4:dC27 acyl sucrose), C₃₉H₆₈O₁₅ (S4:C27 acyl sucrose), C₃₈H₆₆O₁₅ (S4:C26 acyl sucrose), and C₄₀H₇₀O₁₅ (S4:C28 acyl sucrose) in comparison to susceptible plants that are not able to produce the said set of S4 sugars or they just produce tri acylsucroses (S3). Marker M1 is 100% linked with the type of acyl sugars, and one specific sequence was mapped on chromosome 11, which encoded a member of the BAHD family of acyltransferases, more specifically an acetyl-CoA-dependent acyltransferase enzyme ASAT3, capable of acyl sucrose acetylation and linked to the production of C₃₉H₆₆O₁₅ (S4:dC27 acyl sucrose), C₃₉H₆₈O₁₅ (S4:C27 acyl sucrose), C₃₈H₆₆O₁₅ (S4:C26 acyl sucrose), and C₄₀H₇₀O₁₅ (S4:C28 acyl sucrose).

Sequencing of the functional ASAT3 gene resulted in a genomic sequence including promotor region. Plants comprising SEQ ID No. 1, i.e. a functional ASAT3, in combination with AP2e have an increased acyl sugar content and are more resistant to whitefly compared to the plants without SEQ ID No. 1. SEQ ID No.1 shows the coding sequence of ASAT3 in the plant of present invention that encodes for the ASAT3 protein of SEQ ID No. 2. SEQ ID No.3 shows the coding sequence of AP2e in the plant of present invention that encodes for the AP2e protein of SEQ ID No. 4.

TABLE 1
Marker sequences used for QTL mapping

| Marker | Sequence | SEQ ID No. |
|---|---|---|
| M5_F | GCGAGGCATTTGTTGAAGTTGCTAATGC | 7 |
| M5_R | GGTTGATACAAACAGCCCATTG | 8 |
| M1_F | GCCTTCTTCTACCCTAAAATAC | 9 |
| M1_R | CAGCTAAGGGATAATAAAAGGAC | 10 |

It was found that the ASAT3 gene in combination with the AP2e gene specifically promotes and regulates the production of specific types of S4 acyl sugars C₃₉H₆₆O₁₅, C₃₉H₆₈O₁₅, C₃₈H₆₆O₁₅, and C₄₀H₇₀O₁₅. The combination AP2e (marker M5)+ASAT3 (marker M1) increases the level of specific S4 acyl sucroses that are needed for whitefly resistance. Several tomato plants were selected for their presence of AP2e gene and the presence/absence of ASAT3 gene, homo/heterozygous using the M5 and M1 markers. Total acyl sugars content (g per gram plant fresh weight (gFW) per plant was determined, as well as the presence of specific acyl sugars C₃₉H₆₆O₁₅, C₃₉H₆₈O₁₅, C₃₈H₆₆O₁₅, and C₄₀H₇₀O₁₅, and the resistance to whitefly. The genotypes of ASAT3 are co-segregating with the production of C₃₉H₆₆O₁₅, C₃₉H₆₈O₁₅, C₃₈H₆₆O₁₅, and C₄₀H₇₀O₁₅ acyl sucrose, and is linked with the white fly resistance level, see Table 2.

TABLE 2
AP2e, ASAT3 presence in plants and the effect
on Acyl sugars and whitefly resistance.

| | | | | Sum S4C26 + | |
|---|---|---|---|---|---|
| | | | Total | S4C27 + S4:dC27 + | % WF |
| Plant | AP2e | ASAT3 | AcS(μg/gFW) | S4C28 (μg/gFW) | Toxicity |
| 1 | b | a | 1814.99 | 17.46 | 38.1 |
| 2 | b | a | 858.12 | 5.75 | 30 |
| 3 | b | a | 2060.41 | 17.01 | 45 |
| 4 | b | a | 985.78 | 7.00 | 48.7 |
| 5 | b | h | 2662.10 | 630.51 | 85.8 |
| 6 | b | h | 2455.59 | 548.57 | 94.4 |
| 7 | b | h | 2993.71 | 603.38 | 85.2 |
| 8 | b | h | 2482.14 | 530.20 | 85.1 |
| 9 | b | b | 2825.60 | 762.10 | 82.8 |
| 10 | b | b | 1863.18 | 536.83 | 75.7 |
| 11 | b | b | 2128.93 | 613.07 | 71.2 |
| 12 | b | b | 1389.11 | 395.52 | 70 |

a = absent
h = heterozygous
b = present

Furthermore, in an additional experiment and to confirm the above results, the whitefly toxicity levels and average amount of C₃₉H₆₆O₁₅, C₃₉H₆₈O₁₅, C₃₈H₆₆O₁₅, and C₄₀H₇₀O₁₅ acyl sugar were determined as indicated above in plants that comprise the ASAT3 gene homozygous (+/+) or heterozygous (+/−), or in case the ASAT3 gene is absent (−/−). FIG. 3A shows that the relative whitefly (WF) toxicity in the plants comprising the ASAT3 gene outperforms the plants absent of ASAT3, wherein the homozygous plants provide the most optimal WF resistant plant. FIG. 3B shows that the average sum of C₃₉H₆₆O₁₅, C₃₉H₆₈O₁₅, C₃₈H₆₆O₁₅, and C₄₀H₇₀O₁₅ acyl sugar (indicated as S4 type acyl sugar) directly linked to the presence or absence of the ASAT3 gene, wherein the homozygous plants produce the highest average amount of said specific sugars. As long as ASAT3 gene in combination with the AP2e gene is present, the plants are more resistant to whitefly and also seem to be more productive in terms of C₃₉H₆₆O₁₅, C₃₉H₆₈O₁₅, C₃₈H₆₆O₁₅, and C₄₀H₇₀O₁₅ acyl sugars.

### AP2e and ASAT3 Gene Expression Levels and WF Resistance in Tomato

AP2e and ASAT3 gene expression levels were determined in whitefly resistance and susceptible tomato plants. Three types of plants were included; Plant A is homozygous (+/+) for the AP2e gene and heterozygous (+/−) for the ASAT3 gene, Plant B is heterozygous for both AP2e and ASAT3, Plant C is absent (−/−) of AP2e and heterozygous for ASAT3. Furthermore, the acylsugar content of the specific S4 acyl sugars C₃₉H₆₆O₁₅, C₃₉H₆₈O₁₅, C₃₈H₆₆O₁₅, and C₄₀H₇₀O₁₅ was determined and whitefly resistance was determined as described earlier. See Table 3 for the summarized results per plant. Plants homozygous for AP2e and heterozygous for ASAT3 produce the highest amount of the said acylsugars C₃₉H₆₆O₁₅, C₃₉H₆₈O₁₅, C₃₈H₆₆O₁₅, and C₄₀H₇₀O₁₅ and therefore they are more toxic for WF. On the other hand, plants heterozygous for both genes produce less C₃₉H₆₆O₁₅, C₃₉H₆₈O₁₅, C₃₈H₆₆O₁₅, and C₄₀H₇₀O₁₅ acylsugars and for this reason they show a lower WF toxicity.

TABLE 3
AP2e and ASAT3 provide insect resistance in tomato.

| | | | Sum S4C26 + | |
|---|---|---|---|---|
| | | | S4C27 + S4:dC27 + | |
| | AP2e | ASAT3 | S4C28 (μg/g FW) | % WF Toxicity |
| Plant A | +/+ | +/− | 705.23 | 88.92 |
| Plant B | +/− | +/− | 33.13 | 45.26 |
| Plant C | −/− | +/− | 2.74 | 7.45 |

RNA was extracted from the plants. Briefly, several stem pieces from each plant were placed inside a 50 ml tube and subsequently frozen in liquid nitrogen. Next, tubes were vortexed removing the trichomes from the samples, which are afterwards located at the bottom of the tube. Then, the tubes are placed back in liquid nitrogen. RNA extraction was performed using the RNeasy Plant Mini Kit from Quiagen following manufacturer's instructions. RNA quantity was measured by using nanodrop from Quiagen.

Next, 1 μg of RNA was DNAase-treated using DNase I from Thermo Scientific following manufacturer's instructions. RNA was reverse transcribed using the High-Capacity cDNA Reverse Transcription Kit from Applied Biosystems following manufacturer's instructions. For the reverse transcription reaction, total RNA was divided into two independent reactions. 500 ng for a reaction containing the reverse transcriptase and the other 500 ng for a reaction lacking the enzyme as negative control. The cDNA produced was diluted 3× adding 40 μl of nuclease-free water.

1 μl of cDNA was loaded in a qPCR reaction with 10 μl of Go-Taq qPCR Master Mix from Promega, 0.4 μl of each primer (Table 4) 10 μM and with nuclease-free water filling up to 20 μl final volume. For each primer pair, relative standard curves were also made by performing 2× successive dilutions starting from the original 3× diluted cDNA. Expression analysis was performed using the ΔΔCt method using β-Actin as endogenous control. Expression values of each gene were normalized to the endogenous β-Actin gene (FIG. 4).

TABLE 4
RT-qPCR primer sequences

| Primer Name | Primer sequence | SEQ ID No. |
|---|---|---|
| AP2e Fw | GCGGATAATACAACAGCAAC | 11 |
| AP2e Rev | CTGTGAACTTCTTGACCTTG | 12 |
| β-Actin Fw | AAAAGTGCGAGTGTCCTGTCT | 13 |
| β-Actin Rev | TCAAAAAAACAAATTGACTGG | 14 |
| ASAT3 Fw | GTGAACCTGTAAGAGTAACC | 15 |
| ASAT3 Rev | TGATAAGTACATTTAGCCCATC | 16 |

1. A tomato plant having improved whitefly resistance, wherein said plant comprises a combination of an acetyl-CoA-dependent acyltransferase gene (ASAT3) encoding a cDNA sequence having at least 95% sequence identity with SEQ ID No. 1, and an APETALA2e ethylene-responsive transcription factor gene (AP2e) encoding a cDNA sequence having at least 95% sequence identity with SEQ ID No. 3, wherein said combination of ASAT3 and AP2e genes result in an increased acyl sugar content of one or more sugars selected from the group consisting of C₃₉H₆₆O₁₅, C₃₉H₆₈O₁₅, C₃₈H₆₆O₁₅, C₄₀H₇₀O₁₅, preferably all of said sugars, as compared to a tomato plant not comprising said combination of genes.

2. The tomato plant according to claim 1, wherein the ASAT3 gene encodes for the protein sequence represented by SEQ ID No. 2, and wherein the AP2e gene encodes for the protein sequence represented by SEQ ID No. 4.

3. The tomato plant according to claim 1, wherein the acyl sugar content of C₃₉H₆₆O₁₅, C₃₉H₆₈O₁₅, C₃₈H₆₆O₁₅, and C₄₀H₇₀O₁₅ together is at least 200 μg/g of fresh weight (FW) of plant leaves, preferably at least 250 μg/g of fresh weight (FW) of plant leaves, more preferably at least 300 μg/g of fresh weight (FW) of plant leaves.

4. The tomato plant according to claim 1, wherein the acyl sugar content of C₃₉H₆₆O₁₅ is at least 1 μg/g of FW of plant leaves, preferably at least 1.5 μg/g of FW of plant leaves, more preferably at least 2 μg/g of FW of plant leaves, and/or wherein the acyl sugar content of C₃₉H₆₈O₁₅ is at least 200 μg/g of FW of plant leaves, preferably at least 250 μg/g of FW of plant leaves, more preferably at least 300 μg/g of FW of plant leaves, and/or wherein the acyl sugar content of C₃₈H₆₆O₁₅ is at least 5 μg/g of FW of plant leaves, preferably at least 10 μg/g of FW of plant leaves, more preferably at least 15 μg/g of FW of plant leaves, and/or wherein the acyl sugar content of C₄₀H₇₀O₁₅ is at least 5 μg/g of FW of plant leaves, preferably at least 10 μg/g of FW of plant leaves, more preferably at least 15 μg/g of FW of plant leaves.

5. (canceled)

6. (canceled)

7. (canceled)

8. The tomato plant according to claim 1, wherein the plant is obtainable from deposit NCIMB 44054.

9. The tomato plant according to claim 1, wherein the plant is furthermore resistant to mite, preferably spider mite (Tetranychus urticae).

10. The tomato plant according to claim 1, wherein the ASAT3 gene is at least heterozygous present in the genome of the plant, preferably homozygous.

11. The tomato plant according to claim 1, wherein the AP2e gene is at least heterozygous present in the genome of the plant, preferably homozygous.

12. The tomato plant according to claim 1, wherein said plant is a Solanum lycopersicum var. cerasiforme.

13. The tomato plant according to claim 1, wherein said plant does not comprise a SlAT2 gene in its genome encoding a cDNA sequence with SEQ ID No. 5.

14. The tomato plant according to claim 1, wherein the combination of ASAT3 and AP2e genes furthermore result in an increased acyl sugar content of C₃₂H₅₄O₁₅ as compared to a tomato plant not comprising said combination of genes.

15. A seed, fruit or plant part of a tomato plant according to claim 1.

16. A method for providing a tomato plant having improved whitefly resistance, the method comprises the steps of providing a whitefly susceptible tomato plant and mutating its genome comprising;
providing a combination of an acetyl-CoA-dependent acyltransferase gene (ASAT3) encoding a cDNA sequence having at least 95% sequence identity with SEQ ID No. 1, and an APETALA2e ethylene-responsive transcription factor gene (AP2e) encoding a cDNA sequence having at least 95% sequence identity with SEQ ID No. 3, wherein said combination of ASAT3 and AP2e genes result in an increased of one or more of C₃₉H₆₆O₁₅, C₃₉H₆₈O₁₅, C₃₈H₆₆O₁₅, C₄₀H₇₀O₁₅ acyl sugar content as compared to a tomato plant not comprising said combination of genes.

17. A method for selecting a tomato plant having improved whitefly resistance, wherein the method comprises the steps of;
a) crossing of a tomato plant that is susceptible to whitefly with a tomato plant according to any of the claim 1,
b) selecting S. lycopersicum plants having improved insect resistance that comprise the AP2e gene and ASAT3 gene.

18. The method according to claim 17, wherein presence of the AP2e gene in said S. lycopersicum plants having improved insect resistance is determined by using markers SEQ ID No. 7 and SEQ ID No. 8, and wherein presence of the ASAT3 gene is determined by using markers SEQ ID No. 9 and SEQ ID No. 10.

19. The method according to claim 17, wherein the selection of S. lycopersicum plants having improved insect resistance is by determination of C₃₉H₆₆O₁₅, C₃₉H₆₈O₁₅, C₃₈H₆₆O₁₅, and/or C₄₀H₇₀O₁₅ acyl sugar content, wherein the acyl sugar content of C₃₉H₆₆O₁₅, C₃₉H₆₈O₁₅, C₃₈H₆₆O₁₅, and C₄₀H₇₀O₁₅ together is at least 200 μg/g of fresh weight (FW) of plant leaves, and/or of C₃₉H₆₆O₁₅ is at least 1 μg/g of FW of plant leaves, and/or of C₃₉H₆₈O₁₅ is at least 200 μg/g of FW of plant leaves, and/or of C₃₈H₆₆O₁₅ is at least 5 μg/g of FW of plant leaves.

20. A method for providing a tomato plant having improved whitefly resistance, wherein the method comprises the steps of
a) providing a tomato plant according to claim 1, comprising the AP2e and ASAT3 genes,
b) crossing the tomato plant of step a) with a tomato plant that is more susceptible to whitefly not comprising the AP2e and ASAT3 genes,
c) optionally, selfing the plant obtained in step b) for at least one time,
d) selecting the plants having improved whitefly resistance.

21. A combination of an acetyl-CoA-dependent acyltransferase gene (ASAT3) encoding a cDNA sequence having at least 95% sequence identity with SEQ ID No. 1, and an APETALA2e ethylene-responsive transcription factor gene (AP2e) encoding a cDNA sequence having at least 95% sequence identity with SEQ ID No. 3 for providing insect resistance in tomato plants.

22. Use of a combination of ASAT3 gene and AP2e gene according to claim 21 in tomato plants for providing whitefly resistant tomato plants.


SEQUENCE LISTING (16 sequences)

SEQ ID NO: 1 - DNA, 1293 bp, Solanum lycopersicum
  source 1..1293: mol_type = other DNA
atggcatcatcaacaattatatctagaaaaatgattaaacttttatccccaactccatcttcacttagatgtcacaaactctctttcatggatcacataaatttccctctacattctccatatgccttcttctaccctaaaatacctcaaaattatagtaacaaaatatcacaaatacttgaaaattccctttcaaaagtattgtccttttattatcccttagctggaaaaatcaataataattatacctacgtcgattgtaacgacacaggtgctgagtatttaaacgtccgtatagattgtccaatgtctcaaattctcaaccacccttataacgatgttgtggatgtagttttcccacaagatttgccttggagtagtagtagcttgactcgaagtccactagtggttcaattaagccattttgattgtggtggagttgcagtgagtgcatgtacatcacatacaatttttgatggatattgtctctctaaattcataaacgattgggcgtctacagctcgaaacatggatttcaacccatctcctcagttcaatgcatcaactttcttccctttaccgtctgaaactaatttgagtagcaccctacctgcaacgcgtccatcacaacgtcatgtctcaagaatgtataacttctcatcctcgaatttgacaagactcaaggacatcgtaacaaaagaatcacatgtgaagaatccaactcgcattgaagttgcctcagcacttgttcataaatgtggggtgactatgtcaatggagagttcaggcatgttcaaaccaactctgatgagccatgctatgaatttacgcccaccaattccactgaacacaatgggaaatgcaacatgtatcattctcacaacagcaatgacagaagatgaggtaaaacttccaaactttgttgctaaactacagaaggataaacaacaacttcgagacaagttgaaggatatgaaagaagataggatgcccttatatacacttgaactaggtaaaaacgcgatgaacataatagagaaggatacacatgatgtttatctttgctcaggcatgaccaatactggattacataagatcgatttcggatggggtgaacctgtaagagtaaccctagcaacacatccaaagaagaacaacttcattttcatggatgaacaaagtggagatgggctaaatgtatttatcactttaacaaaagatgatatgctcaagtttcagagcaacaaggagcttctggagtttgcttctccagttgttgaatcaaccaaataa

SEQ ID NO: 2 - protein, 430 aa, Solanum lycopersicum
MASSTIISRKMIKLLSPTPSSLRCHKLSFMDHINFPLHSPYAFFYPKIPQNYSNKISQILENSLSKVLSFYYPLAGKINNNYTYVDCNDTGAEYLNVRIDCPMSQILNHPYNDVVDVVFPQDLPWSSSSLTRSPLVVQLSHFDCGGVAVSACTSHTIFDGYCLSKFINDWASTARNMDFNPSPQFNASTFFPLPSETNLSSTLPATRPSQRHVSRMYNFSSSNLTRLKDIVTKESHVKNPTRIEVASALVHKCGVTMSMESSGMFKPTLMSHAMNLRPPIPLNTMGNATCIILTTAMTEDEVKLPNFVAKLQKDKQQLRDKLKDMKEDRMPLYTLELGKNAMNIIEKDTHDVYLCSGMTNTGLHKIDFGWGEPVRVTLATHPKKNNFIFMDEQSGDGLNVFITLTKDDMLKFQSNKELLEFASPVVESTK

SEQ ID NO: 3 - DNA, 1425 bp, Solanum lycopersicum
  source 1..1425: mol_type = other DNA
atgttggatctcaatgtaagcgtaatctacagtaatgaccttccacaagtttctctacttgatgaatcagccacctccaattcatccttacgaaatgcggaagctacaaccagtgccggtgacgaagattcgtgcgccggtgagttgttcgctttcaattttggaatcctcaaagttgaaggagctgagactagtaggagcagcaacaacgatgatgaggaagcatacggtaagaatcagagagttactcattctcaattcgtgactaggcagctgtttcccgttgatgatggtgagttgaaccggaaacaaaccgatcgggtcattctctcctccgctcgatccggtacttctatcggttttggagatgtgcggataatacaacagcaacaaacggagcaaccgaaacaacaagtgaagaagagtaggagaggcccaaggtcaagaagttcacagtacagaggtgtcactttctaccgtagaactggtagatgggaatcacatatatgggactgtgggaaacaagtatatttgggtggttttgatactgctcacacagcagcaagagcttatgacagagctgcaattaaatttaggggtgttgatgctgatatcaactttagcttaagtgattacgaggaggatatgcaacagatgaaaaaccttggtaaagaagaatttgtgcacttgctgcgacgccatagcactggtttctcaagagggagctccaaattcagaggagtgacgctacataaatgtggcagatgggaggctcggatgggacagttcctcgggaaaaagtatatatatcttgggctgttcgacagcgaagtagaagctgcaagggcctacgataaggcggcaattaaaactagcggaagggaagctgttaccaactttgagccaagtagctatgaaggggaaacaatgtctttaccacagagtgaaggtagccaacatgatcttgatctgaacttggggatatcgaccacttcttcaaaggaaaatgacaggttgggaggttctcgctatcatccttacgatatgcaagacgcaacaaaacctaagatggataaacctggttcagtaatagttggaagttcacatctcaagggactatcaatgtcgtcccaacaagctcaattgtggactggaatctattctaatttctcttccagctatgagggaagagcatatgacaagagaaaggacacaggttcatcacaaggacctccaaattgggcattgcaaatgcctagtcaggttgatacaaacagcccattgacaatgttctgcacggcatcatcatcaggattcttcattccatctactacttctgtcacttcatcaacatctgcattagcaacttcaacaaatgcctcgcagtgcttttaccagattaatccccgcctaccacttccataa

SEQ ID NO: 4 - protein, 474 aa, Solanum lycopersicum
MLDLNVSVIYSNDLPQVSLLDESATSNSSLRNAEATTSAGDEDSCAGELFAFNFGILKVEGAETSRSSNNDDEEAYGKNQRVTHSQFVTRQLFPVDDGELNRKQTDRVILSSARSGTSIGFGDVRIIQQQQTEQPKQQVKKSRRGPRSRSSQYRGVTFYRRTGRWESHIWDCGKQVYLGGFDTAHTAARAYDRAAIKFRGVDADINFSLSDYEEDMQQMKNLGKEEFVHLLRRHSTGFSRGSSKFRGVTLHKCGRWEARMGQFLGKKYIYLGLFDSEVEAARAYDKAAIKTSGREAVTNFEPSSYEGETMSLPQSEGSQHDLDLNLGISTTSSKENDRLGGSRYHPYDMQDATKPKMDKPGSVIVGSSHLKGLSMSSQQAQLWTGIYSNFSSSYEGRAYDKRKDTGSSQGPPNWALQMPSQVDTNSPLTMFCTASSSGFFIPSTTSVTSSTSALATSTNASQCFYQINPRLPLP

SEQ ID NO: 5 - DNA, 1356 bp, Solanum lycopersicum
  source 1..1356: mol_type = other DNA
atgaattgttatattgaaattcaatcaaggaaaatggtgaaaccctcagctcctaccccggataatcttcggagattgaagctttccttgttcgatcagatggatattggtgcatatgtaccaattgtcttcaactacttgccgaacagcacttcatcatatgatcatgatgataagcttgaaaaatcattgtcggagacgctaaccaagttttacccttttgctggaagatttagaaaaggcattgatccattttccatcgactgcaatgatgaaggtattgaatatgttcgaaccaaagtcaatgcagacgatcttgcccaatatctccgtggtcaagcccataatgatattgagtcgtctttgattgatcttcttcctgtaatgcatcgtctaccatcaagtccattatttggtgttcaagtgaatgtattcaataacggaggtgtaaccatagggatacaaattttacatatggtatctgatgctttcactttagtaaaatttgtaaatgaatgggcgcacaccacccttacagggacgatgccactagataatcccggttttggtcaattgccatggctatttccagcaagagcgctaccgtttccattacctgatttcaacactactactgcccctaattataagaatgttacaaagaggtttctctttgatgctttggcaatagaaaacctcagaaatacaatcaaagccaatgacatgatgatgaagcaaccttctagagtggtggtcgtgatgtccctaatatggaaggttcttacacacatttcttccgccaaaaataatggaaattcaagggactcatctttagtgtttgttgttaatttgaggggaaaactgtcatgtactgcaccgtctttagaacacgttgtagggaattgtgtaataccagcaactgctaacaaggagggcgatgaggcaagaagaaaggatgatgagttgaatgatttcgttaagttggtaagaaatacaatacgggacacatgtgaagccattggtaaggcggaaagcgttgatgatatttcctctttagcatttaacaatctgacgaaatgtatagaaaaaattctgcatggagacgagatggacttctattcgtgctctagttggtgcggattcccttggtatgaagctgactttggttggggaaagccattctgggtgagctcagttagttttggtcatcatggagtaactaatctcatggacacaaaagatggtgatggaatacaagtaacaatttgtttgaaggagaatgacatgattgagtttgagagagaccctcacattttgtcctccacttcaaaactagcattccattccttaggataa

SEQ ID NO: 6 - protein, 451 aa, Solanum lycopersicum
MNCYIEIQSRKMVKPSAPTPDNLRRLKLSLFDQMDIGAYVPIVFNYLPNSTSSYDHDDKLEKSLSETLTKFYPFAGRFRKGIDPFSIDCNDEGIEYVRTKVNADDLAQYLRGQAHNDIESSLIDLLPVMHRLPSSPLFGVQVNVFNNGGVTIGIQILHMVSDAFTLVKFVNEWAHTTLTGTMPLDNPGFGQLPWLFPARALPFPLPDFNTTTAPNYKNVTKRFLFDALAIENLRNTIKANDMMMKQPSRVVVVMSLIWKVLTHISSAKNNGNSRDSSLVFVVNLRGKLSCTAPSLEHVVGNCVIPATANKEGDEARRKDDELNDFVKLVRNTIRDTCEAIGKAESVDDISSLAFNNLTKCIEKILHGDEMDFYSCSSWCGFPWYEADFGWGKPFWVSSVSFGHHGVTNLMDTKDGDGIQVTICLKENDMIEFERDPHILSSTSKLAFHSLG

SEQ ID NO: 7 - DNA, 28 bp, unidentified
  source 1..28: mol_type = other DNA
gcgaggcatttgttgaagttgctaatgc

SEQ ID NO: 8 - DNA, 22 bp, unidentified
  source 1..22: mol_type = other DNA
ggttgatacaaacagcccattg

SEQ ID NO: 9 - DNA, 22 bp, unidentified
  source 1..22: mol_type = other DNA
gccttcttctaccctaaaatac

SEQ ID NO: 10 - DNA, 23 bp, unidentified
  source 1..23: mol_type = other DNA
cagctaagggataataaaaggac

SEQ ID NO: 11 - DNA, 20 bp, unidentified
  source 1..20: mol_type = other DNA
gcggataatacaacagcaac

SEQ ID NO: 12 - DNA, 20 bp, unidentified
  source 1..20: mol_type = other DNA
ctgtgaacttcttgaccttg

SEQ ID NO: 13 - DNA, 21 bp, unidentified
  source 1..21: mol_type = other DNA
aaaagtgcgagtgtcctgtct

SEQ ID NO: 14 - DNA, 21 bp, unidentified
  source 1..21: mol_type = other DNA
tcaaaaaaacaaattgactgg

SEQ ID NO: 15 - DNA, 20 bp, unidentified
  source 1..20: mol_type = other DNA
gtgaacctgtaagagtaacc

SEQ ID NO: 16 - DNA, 22 bp, unidentified
  source 1..22: mol_type = other DNA
tgataagtacatttagcccatc